(19) United States
(12) Patent Application Publication
(10) Pub. No.: US 2008/0300485 A1
(43) Pub. Date: Dec. 4, 2008

(54) ULTRASONIC SYSTEM AND METHOD FOR MEASUREMENT OF OCULAR BIOMECHANICS

(75) Inventors: Jun Liu, Columbus, OH (US); Cynthia J. Roberts, Columbus, OH (US)

Correspondence Address: DINSMORE & SHOHL LLP, ONE DAYTON CENTRE, ONE SOUTH MAIN STREET, SUITE 1300, DAYTON, OH, 45402-2023, US

(73) Assignee: THE OHIO STATE UNIVERSITY, Columbus, OH (US)

(21) Appl. No.: 12/110,381

(22) Filed: Apr. 28, 2008

Related U.S. Application Data
(60) Provisional application No. 60/914,368, filed on Apr. 27, 2007.

(51) Int. Cl. A61B 8/00

(52) U.S. Cl. 600/438

(57) ABSTRACT

A system and a method for non-invasively ultrasonically measuring biomechanical properties of ocular tissue in vivo is presented. The method comprises positioning an ultrasonic transducer proximally to the ocular tissue. Reflections of the ocular tissue can be ultrasonically obtained using the ultrasonic transducer. The ultrasonic reflections can be converted into reflection spectra. Biomechanical properties of the ocular tissue, such as, for example, thickness, corneal stiffness, density, and longitudinal modulus, can be determined based on the reflection spectra. A wave propagation model can be developed to simulate ultrasound propagation of ocular tissue in vivo. The ultrasonic non-destructive evaluation method and system for the non-invasive measuring of reflection spectra and determining biomechanical properties of ocular tissue in vivo can provide information for ocular disease management and therapeutic and refractive procedures.

### CROSS-REFERENCE TO RELATED APPLICATIONS

This application claims the benefit of U.S. Provisional Application Ser. No. 60/914,368 (OSU 0049 MA), filed Apr. 27, 2007.

### BACKGROUND OF THE INVENTION

The present invention relates to an ultrasonic method and system for non-invasively measuring and determining biomechanical properties of ocular tissue.

The fluid pressure inside the eye (i.e., the intraocular pressure or IOP) is responsible for maintaining the positions of various intraocular structures in order to achieve visual acuity. This pressure presents a mechanical loading to ocular tissue, and the mechanical responses of ocular tissue to this loading are dependent on their innate biomechanical properties. However, current commercial ultrasound methods of measuring corneal thickness, or pachymetry, typically assumes a single speed of sound for all human cornea of 1640 m/s.

In addition, the biomechanical properties of corneal tissue are essential for the eye's normal physiological function, i.e., maintaining a spherical shape for visual acuity. These properties may be altered by either disease or surgical operations. Studies have shown that keratoconus, a corneal disease that manifests as bulging of corneal tissue around an apex, is correlated with corneal thinning and softening. Ablative corneal surgery, a refractive procedure in which corneal tissue is removed in a specific pattern to correct myopia or hyperopia, may also introduce changes in biomechanical properties. Studies have also shown that the biomechanical responses of ocular tissue may play an important role in pathogenesis of multiple ocular diseases (e.g., glaucoma). Variations in corneal biomechanical properties may be a significant confounding factor for tonometry measurement of IOP, a routine practice for glaucoma screening. Non-invasive determination of corneal mechanical properties is therefore important for detection and monitoring of ocular diseases.

The elastic modulus of ex vivo cornea tissue has been studied in the past. However, currently, no devices can non-invasively measure corneal elasticity in vivo. Therefore, there is a need for an ultrasonic method and system to non-invasively measure and determine the biomechanical properties of ocular tissue in vivo using ultrasound spectroscopic methods.

### BRIEF SUMMARY OF THE INVENTION

According to the present invention, an ultrasonic non-destructive evaluation method and system for the non-invasive measuring of biomechanical properties of ocular tissue in vivo is presented. The method comprises positioning an ultrasonic transducer proximally to the ocular tissue. Reflections of the ocular tissue can be ultrasonically obtained using the ultrasonic transducer. The ultrasonic reflections can be converted into reflection spectra. Biomechanical properties of the ocular tissue, such as, for example, thickness, stiffness, density, and longitudinal modulus, can be determined based on the reflection spectra. A wave propagation model can be developed using the measured biomechanical properties. The measured biomechanical properties can provide information for ocular disease management and therapeutic and refractive procedures.

Accordingly, it is a feature of the embodiments of the present invention to provide an ultrasonic non-destructive evaluation method and system for the non-invasive measuring of biomechanical properties of ocular tissue in vivo.

It is another feature of the embodiments of the present invention to provide a wave propagation model to assist in the ultrasonic non-destructive evaluation method for the non-invasive measuring of biomechanical properties of ocular tissue in vivo. Other features of the embodiments of the present invention will be apparent in light of the description of the invention embodied herein.

### BRIEF DESCRIPTION OF THE SEVERAL VIEWS OF THE DRAWINGS

The following detailed description of specific embodiments of the present invention can be best understood when read in conjunction with the following drawings, where like structure is indicated with like reference numerals and in which:

FIG. 1 illustrates plane wave propagation in a thin layer structure of cornea or contact lens according to an embodiment of the present invention.

FIG. 2 illustrates a schematic of the measurement and signal processing system according to an embodiment of the present invention.

FIG. 3A-C are graphs of the effects of different physical parameters on the overall reflection spectra predicted by the wave propagation model, according to an embodiment of the present invention.

FIG. 4 illustrates the measured ultrasonic reflection spectra from three types of contact lenses according to an embodiment of the present invention.

FIG. 5 is a graph representing the comparison of experimental and reconstructed reflection spectra according to an embodiment of the present invention.

FIG. 6 is a graph representing the comparison of thickness measured directly by an electronic thickness gauge and reconstructed from the ultrasonic method according to an embodiment of the present invention.

FIG. 7 is a graph representing human corneal stiffness results according to an embodiment of the present invention.

### DETAILED DESCRIPTION

In the following detailed description of the embodiments, reference is made to the accompanying drawings that form a part hereof, and in which are shown by way of illustration, and not by way of limitation, specific embodiments in which the invention may be practiced. It is to be understood that other embodiments may be utilized and that logical, mechanical and electrical changes may be made without departing from the spirit and scope of the present invention.

A method and system for measurements of ocular tissue, such as, for example, human corneas, can be developed using ultrasound spectroscopic methods. Human corneas present a unique opportunity for the application of ultrasonic techniques due to their direct accessibility and structural simplicity. In the past, ultrasound spectroscopy typically has been used to characterize a thin layer of non-biological material embedded between two substrates. The resulting spectra of the ultrasonic reflections from this thin layer tend to be dependent on a set of layer material properties such as density, thickness and elastic moduli. However, the cornea can be treated as a homogenous layer for ultrasonic modeling and property reconstruction. This ultrasonic approach can be validated in terms of accuracy of the estimated properties by first performing the measurements on soft contact lens to avoid unknown confounding factors associated with biological tissue samples (e.g., hydration status of corneas). The ultrasonic estimation of the properties can then be compared with those estimations obtained by standard methods. Then, the method can be tested using human ocular tissue in vivo to determine human corneal stiffness.

### Soft Contact Lens Measurements

Soft contact lenses of three different materials can be used: 1) Hydrogel lenses: Biomedics 55 (Ocular Sciences), 2) Silicone-Hydrogel lenses: Night & Day (CIBA Vision Corp), and 3) Silicone lenses: DuraSoft2 (Wesley Jessen Visioncare). Six lenses, with identical specifications, of each type were used. The lenses were stored in 0.9% saline for more than 24 hours before measurements were taken.

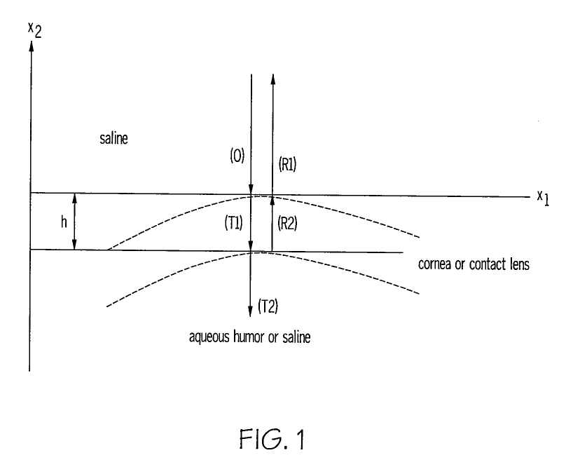

A mathematical model of elastic wave propagation can be constructed to simulate ultrasound propagation of a cornea by using contact lenses immersed in liquid bath. Referring initially to FIG. 1, mechanically, the system can model ocular tissue as a thin layer (i.e., contact lens or cornea) embedded between two continuous subspaces (i.e., saline or saline and aqueous humor). As shown in FIG. 1, the plane wave propagation in a thin layer structure of cornea, or contact lens, can be shown as incident wave (O), the reflected wave in water bath (R1), the transmitted wave in cornea or cornea lens (T1), the reflected wave in cornea, or contact lens, (R2) and the transmitted wave in aqueous humor, or saline, (T2). The thickness of the layer is represented by h.

An elastic wave propagation model can be derived using the system as shown in FIG. 1. A longitudinal time-harmonic wave propagating along the positive direction of x coordinate can be expressed as:

u(x,t)=Ae^{i(kx-ωt)} (1)

where u is the displacement field along x direction, A is the amplitude, ω is the angular frequency, and k is the wave number (k=ω/c, c is wave speed). The stress fields within the layer were derived by using constitutive relationships for isotropic materials.

The reflection coefficient from the thin lens layer can be defined as the ratio between the magnitude of the reflected wave A_{R1} and that of the original wave A_{O} (see FIG. 1). With known properties of the substrates (i.e., saline), and the layer (i.e., thickness h, density ρ, and mechanical properties such as Lame's constants λ and μ), the reflection coefficient can be solved by enforcing continuity conditions at the interfaces between the layer and the substrates. Specifically, the stresses and displacements at the layer-substrate interfaces observe the following equations:

1) Continuity of Stresses

ρ₂₂(0+)=σ₂₂(0−) (2)

σ₂₂(h+)=σ₂₂(h−) (3)

### 2) Continuity of Displacements

u₂(0+)=u₂(0−) (4)

u₂(h+)=u₂(h−) (5)

These four equations (Equations 2-5) can be used to solve for the normalized magnitudes of the four resulting waves R1, R2, T1, and T2, upon the incidence of a known longitudinal wave O (see FIG. 1). By calculating reflection coefficients at a range of frequencies, a reflection spectrum can be obtained. It is noted that the Lame's constants λ and μ appeared in the form of λ+2μ in all equations. Therefore a longitudinal modulus κ can be used to denote λ+2λ.

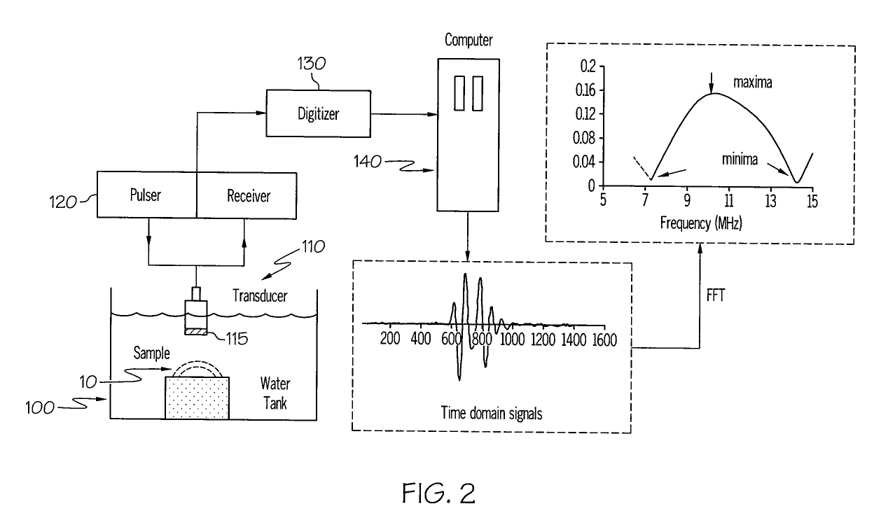

FIG. 2 illustrates a schematic of the measurement and signal processing system according to an embodiment of the present invention. All contact lens samples 10 can be immersed in 0.9% saline liquid bath 100 during ultrasonic measurement. For in vivo measurements, the saline liquid bath 100 for transduction of sound waves may be necessary when using immersion-type ultrasonic transducers 110. In this embodiment, the saline liquid bath 100 can be applied by using an eye cup in the same manner as typically known in ophthalmic ultrasound imaging. In another embodiment, sound waves may be transmitted into the cornea through tear film; however, the near field effect of this type of transducer 110 could make amplitude-dependent measurements difficult due to the variations of acoustic intensity in this region. Additionally, contact-mode transducers can also be used.

A broadband ultrasound transducer 110 can be excited by a pulser-receiver 120. One example of a broadband transducer 110 that can be used is XMS, Panametrics-NDT but any other suitable transducers known in the art may be used. An example of the pulser-receiver 120 can be a 5900PR, Panametrics-NDT. However, any other pulser-receivers 120 known in the art may be used. The X, Y, and Z positions of the transducer 110 can be adjusted using precision linear stages (1 μm step size, Newport) to center the ultrasonic beam to the center of apex of the cornea, or sample contact lens, 10. The distance from the transducer surface 115 to the apex of the sample contact lens 10 can be maintain for all samples. The positioning stages can allow a resolution of about one μm in each direction of adjustment to ensure good positioning of the transducer 110 with respect to the cornea, or sample contact lens, 10.

The ultrasonic reflections can be recorded using a digitizer 130 such as, for example, a DP105, Acqiris, 500 MHz/8-bit. The ultrasonic reflections can be displayed on an output display and stored on a processor 140. The output display and processor 140 can be part of the same device, such as, for example, a typical personal computer. All measurements can be performed under the same pulser-receiver 120 and digitizer 130 settings. The ultrasonic reflections from the cornea, or sample contact lens, 110 layer can be converted by the processor 140 that may be resident on a personal computer into experimental reflection spectra using Fast Fourier Transformation.

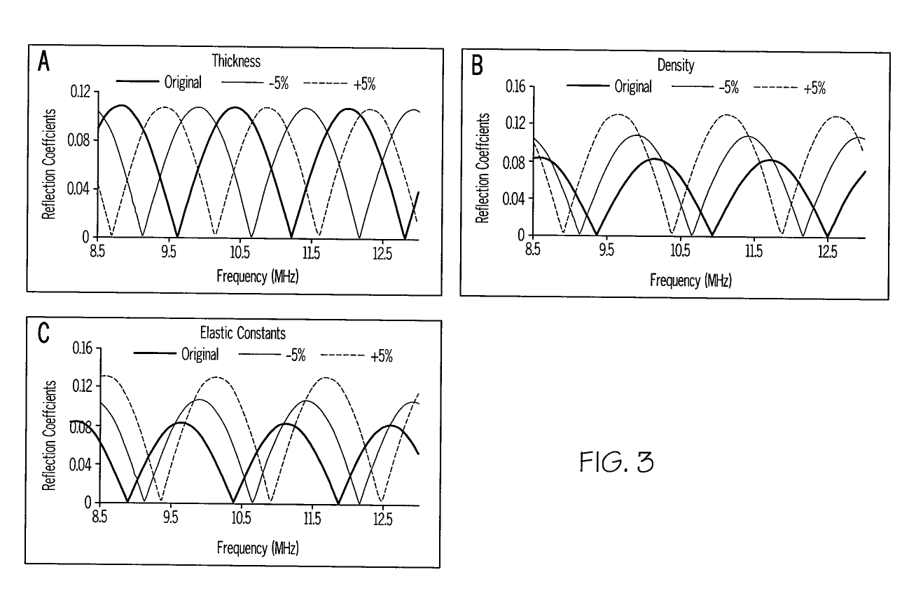

The effects of each material property on the ultrasonic reflection spectra of the thin layer can be simulated. The independent physical properties of the contact lens layer, i.e., density, thickness, and longitudinal modulus, can be varied separately to examine how they affected the characteristics of the reflection spectra. Each parameter can be varied ±5% from an arbitrary original value. The results are shown in FIGS. 3A-C. FIG. 3A illustrates the effect of the different thickness of the sample contact lens, FIG. 3B illustrates the effect of changes in density, and FIG. 3C illustrates the effect of altering the longitudinal modulus. The ultrasonic reflection spectrum has the form of alternating and repeating minima (troughs) and maxima (peaks). The characteristics of the spectral curve can be affected by the material properties of the layer. Referring to FIG. 3A, the changes in thickness alone can affect the locations of the minima and maxima along the frequency axis, but did not affect their magnitudes. Changes in density and elastic constants can change both magnitudes and locations. These two properties can change the magnitudes of the maxima in a similar fashion, yet can have the opposite effect on their frequency locations (See FIGS. 3B and 3C). Variation in any of the material properties can bring forth changes in the characteristics of the reflection spectra. In addition, each property can affect the spectra in a distinct manner.

To summarize derivation of the wave propagation model, the reflection coefficient of the thin layer (defined as the ration between the magnitude of the reflected wave A_{R1} and that of the original wave A_{O}) can be determined by enforcing continuity conditions at the interfaces between the layer and the substrates. By calculating reflection coefficients at a range of frequencies, a reflection spectrum can be obtained. The frequency range can be between about 7 to about 16 MHz. This frequency range can correspond to the bandwidth of the transducer 110 used.

The physical properties (i.e., thickness h, density ρ, and modulus λ+2μ) of the lens layer can be reconstructed using an inverse algorithm. The inverse algorithm can search the multidimensional space to minimize the following function:

ε(h,ρ,κ)=Σ(|R^{e}(f)|−|R^{t}(f)|)², (6)

where m is the number of the data points at different frequencies; R^{e} and R^{t} are the experimental and theoretical reflection coefficients, which are functions of the layer properties and frequency f; ε is the error term that represents the discrepancy between the reconstructed reflection spectra Rt and the experimental reflection spectra R^{e}.

The thickness of each lens can be measured separately using an electronic thickness gauge, such as, for example ET-1 (Rehder), that is commonly used for measuring the thickness of soft contact lenses. Three readings can be taken from each lens and the average can be used to compare with the reconstructed thickness obtained through the ultrasonic method. Direct measurements using the thickness gauge may be biased by the “stiffness” of the samples. As acknowledged by the contact lenses' manufacturers, softer samples are more subject to compression during the measurement; therefore their thickness might be underestimated, while the harder ones overestimated. Indeed, the ultrasonic measurements for the “softer” lenses (Night & Day) were all slightly larger than those measured by the thickness gauge, while the opposite was found for the “harder” lenses (DuraSoft2).

Density of the contact lenses can be measured by comparing the mass of the contact lenses samples in air and in water (Archimedes' principle). Due to their small mass, all six contact lenses of the same type can be scaled together using an analytical balance in air and in water. The density can be calculated as: ρ=(mₐᵢᵣρ_{water}/mₐᵢᵣ−m_{water}), where mₐᵢᵣ and m_{water} were the contact lens mass measured in air and water and ρ_{water}=1.0 g/cm³. Manufacturers' reports on density of the contact lenses were also obtained.

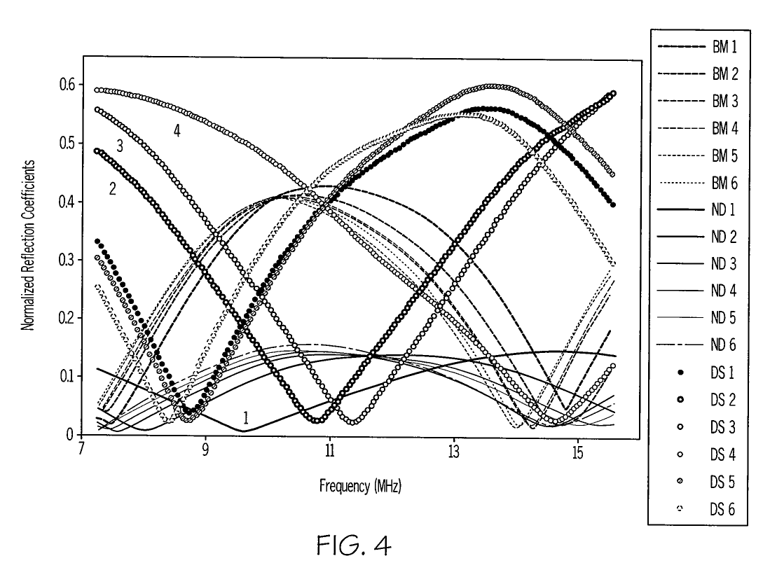

FIG. 4 is a graph representing the ultrasonic spectra from all of the contact lenses measured. The dotted lines represent the Biomedics lenses, the solid lines represent the Night & Day lenses, and the open circles represent the DuraSoft2 lenses. The spectral signals were consistent within the same type lenses in terms of magnitude and distances between maxima and minima. The spectral curves appeared “shifted” along the frequency axis for some of the contact lenses compared to others of the same type (e.g., sample 1 in the Night & Day contact lenses, and samples 2, 3 and 4 in the DuraSoft2 contact lenses). As shown in FIG. 4, the spectra of different material types can differ in terms of the height of the maxima and the distance between adjacent maxima or minima.

Table 1 presents the mean and standard deviation of the reconstructed properties for each type of contact lenses. The Night & Day contact lenses had the lowest reconstructed longitudinal modulus, while the Biomedrics contact lenses had the intermediate, and the DuraSoft2 contact lenses, the highest. Pair-wise Student t-test showed the modulus λ+2μ was significantly different for different types of lenses (P<0.001).

TABLE 1

| | | | Elastic | | | |
|---|---|---|---|---|---|---|
| | Thickness (μm) | | constants^{a} (GPa) | | Density (g/cm³) | |
| | Mean | SD | Mean | SD | Mean | SD |
| Biomedrics | 108.64 | 3.87 | 2.74 | 0.05 | 1.11 | 0.02 |
| Night & Day | 86.14 | 9.72 | 2.14 | 0.23 | 1.09 | 0.01 |
| DuraSoft2 | 76.18 | 15.03 | 2.91 | 0.07 | 1.17 | 0.03 |

^{a}Difference was statistically significant.
P value < 0.001, pair-wise Student t-test.

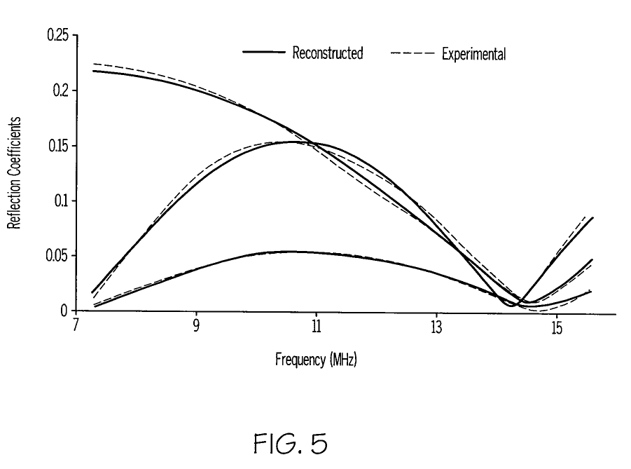

FIG. 5 is a graph that compares the reconstructed and experimental spectra for one contact lens of each group. The reconstructed spectra can be calculated theoretically using the reconstructed properties obtained from the ultrasonic measurements of the respective contact lens.

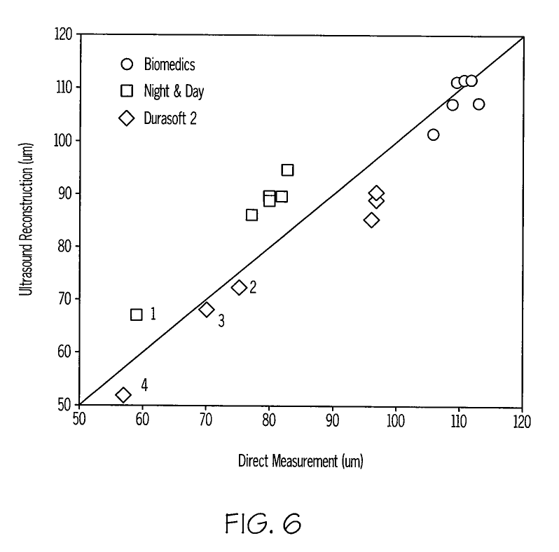

FIG. 6 is a graph of a comparison of the reconstructed and the directly measured thickness for each contact lens. The two measurements were highly correlated (R=92.5%), and all data points were close to the ‘equivalent’ line. Although close to the equivalent, the thickness reconstructed by the ultrasonic method were higher than those obtained using the thickness gauge for the Night & Day contact lenses, while lower for the DurSoft2 contact lenses. Additionally, sample 1 of the Night & Day contact lenses had a very different thickness as compared to the other Night & Day sample contact lenses. The same thickness deviation can be found in samples 2, 3 and 4 of the DuraSoft2 contact lenses.

Table 2 presents a comparison of the reconstructed, the directly measured and reported density of each group of lenses. The differences among these three measurements were small and not statistically significant.

TABLE 2

| | Direct | Ultrasound | Manufacturers' |
|---|---|---|---|
| | measurement | reconstruction | report |
| Biomedics | 1.108 | 1.107 | 1.060 |
| Night & Day | 1.097 | 1.094 | 1.080 |
| DuraSoft2 | 1.203 | 1.174 | 1.176 |

Therefore, the ultrasonic approach was able to differentiate the mechanical properties of the three types of soft contact lenses made of different polymers. The standard deviation was small indicating consistency in measuring samples made of the same material.

The thickness obtained from the ultrasonic method and direct readings from the thickness gauge agreed from sample to sample. The average density readings from ultrasonic reconstruction were consistent with the direct measurements as well as the manufacturers' reports. Therefore, the ultrasonic measurements of two of the three unknown properties (i.e., thickness, density and modulus) can be validated through standard methods. As the graph in FIG. 6 demonstrates, the theoretical spectra calculated from ultrasonic measurements of the properties agreed well with the experimental spectra. Since the theoretical spectra can be uniquely determined by the three properties, it could be inferred that the reconstructed modulus were accurate.

This approach can be applied to in vivo measurements of mammalian ocular tissue, such as, for example, human corneas. However, ultrasound exposure may produce thermal, mechanical or cavitation effects in biological tissues. According to their distinct mechanisms, only thermal effects may be of potential concern in this approach. Thermal effect, which is the absorption of acoustic energy to cause a temperature rise in tissue, can be determined by the spatially and temporally averaged intensity, I_{SPTA,3}, of the transducer output. The FDA 510(k) guideline for ultrasound exposure of ocular tissue is I_{SPTA,3}≦17 mW/cm². The I_{SPTA,3} of the transducer can be far below the threshold due to its unfocused nature. Theoretical estimation based on extrapolation of the reported values can be as low as 0.2 mW/cm². Experimental measurements are needed to obtain actual output characteristics.

Ultrasonic exposure can be further reduced by minimizing the dwelling time of the sound waves on ocular tissue. For example, the transducer can be grossly positioned before it is turned on. It can then be turned on to facilitate fine tuning of the position which takes approximately one to two minutes. After the transducer is positioned, the actual data acquisition can require only a few seconds to complete.

Intraocular pressure loading will likely change the elastic modulus of ocular tissue such as, for example, corneal tissue, due to the intrinsic nonlinearity of the ocular tissue. The nonlinearity of intact ocular tissue can be studied by performing ultrasonic measurements on enucleated eyes with the intraocular pressures maintained and monitored at various levels to provide useful information for constitutive modeling of ocular tissue.

In conclusion, the feasibility of the ultrasonic method and system for the non-destructive evaluation of the mechanical properties of corneal phantoms can be demonstrated. This method and system can be applied to in vivo non-invasive measurement of mammalian ocular tissue, such as, for example, human cornea, human sclera and any other ocular tissues with some adaptation to the algorithm.

### In Vivo Human Corneal Measurements

Using the above results for soft contact lens, an ultrasonic method and system capable of non-invasive characterization of corneal biomechanical properties in vivo can be developed. The method can investigate corneal stiffness and the speed of sound in the cornea of normal human subjects and can determined the potential influence of those variables on the accuracy of current ultrasound pachymeter methods.

Twenty-five normal (i.e., with no known ocular disease present) human subjects were studied. The group comprised sixteen females and nine males. The mean age of the group was 36 years with a standard deviation of ±10 years. The age range of subjects was 23 to 56 years old. The subjects' corneas were measured while the subjects were in the supine position. The corneas were anesthetized by the application of one drop of topical anesthetic, such as, for example, proparacaine. An eye cup filled with buffered saline can be placed on the eye using the method known in the art for a typical A-line ultrasound. A broadband ultrasound transducer with an acoustic intensity (spatial peak, temporal average) of less than 3 mw/cm² can be used to excite the cornea. The ultrasound transducer can be placed in the saline of the eyecup at a set distance from the cornea. The ultrasound transducer can be, for example, a 10 MHz, XMS, Olympus-NDT. In one embodiment, the ultrasound transducer can be placed about 1 cm from the cornea. The system can comprise the ultrasound transducer, a pulser/receiver, a digitizer and a processor as discussed above for measuring the soft contact lens and as seen in FIG. 2.

The linear elastic wave propagation model can be developed as discussed above and can be used to characterize the corneal biomechanical properties. The reflection spectral curves predicted by the wave propagation model were fitted to the experimental measured ultrasonic spectra using a Levernberg-Marquardt nonlinear, least square algorithm to uniquely estimate corneal thickness, density and stiffness. The corneal speed of sound can be calculated using established formulas such as, for example:

$$speed of sound = \sqrt{\frac{stiffness}{density}} \qquad ( 6 )$$

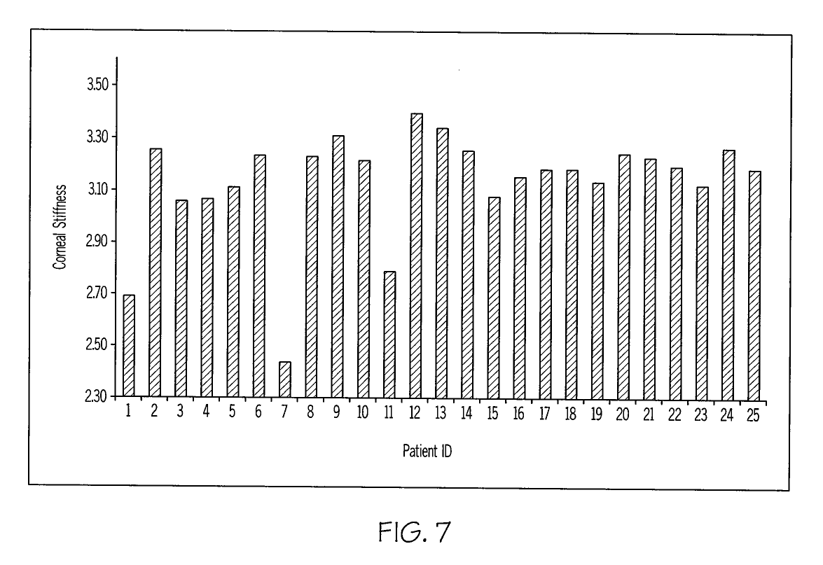

The potential errors in ultrasound pachymetry can be estimated by comparing the corneal thickness based on the measured speed of sound and the assumed speed of sound used in clinical pachymetry (i.e., 1640 m/s). The mean measured speed of sound for the twenty-five subjects was 1636 m/s with a standard deviation of ±49 m/s (with a range of 1476 m/s to 1718 m/s). Turning to FIG. 7, a graph illustrating the corneal stiffness of the twenty-five subjects is shown. The mean corneal stiffness was measured to be 3.14 GPa with a standard deviation of ±0.21 GPa (with a range of 2.44 to 3.40 GPa). A strong correlation between corneal stiffness and the speed of sound (R²=0.77) was evident. For example, for a cornea with an actual thickness of 530 μm, the lower and upper bounds of the corneal speed of sound corresponded to a 59 μm overestimation or a 24 μm underestimation of the corneal thickness in ultrasonic pachymetry measurements. Table 3 lists the measured corneal thickness using three methods: Optical Coherence Tomography (OCT), Ultrasound Pachymetry (US-Pachy) and the experimental model (US-Model).

TABLE 3

| | | Standard |
|---|---|---|
| Method | Mean (μm) | deviation (μm) |
| OCT | 532 | 31 |
| US-Pachy | 536 | 30 |
| US-Model | 534 | 32 |

As can be seen, there was good agreement for the mean value and the standard deviation of corneal thickness in all three methods.

In conclusion, the mean value of the measured corneal speed of sound was found to be consistent with those measurements reported in the literature. Additionally, a large range of corneal speeds of sound was found that was highly correlated with corneal stiffness in the normal human subjects. Therefore, traditional ultrasound pachymetry may significantly overestimate or underestimate corneal thickness owing to the assumptions about the speed of sound in the cornea.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A system for non-invasively ultrasonically measuring biomechanical properties of ocular tissue, the system comprising:
a ultrasonic transducer positioned substantially proximate to the ocular tissue to be measured;
a pulser-receiver to excite the ultrasonic transducer to produce incident ultrasound waves towards the ocular tissue and to receive the ultrasonic reflections back from the ocular tissue; and
a processor to convert the ultrasonic reflections from the pulser-receiver into reflection spectra that are indicative of the biomechanical properties of the ocular tissue, wherein variation of the biomechanical properties alter the reflection spectra distinctly.

2. The system of claim 1, further comprising:
an eye cup filed with saline and positioned against the surface of the ocular tissue, wherein the ultrasonic transducer is positioned within the eye cup.

3. The system of claim 2, wherein the ultrasonic transducer is an immersion-type transducer.

4. The system of claim 1, further comprising:
a digitizer to digitize and record the ultrasonic reflections received from the pulser-receiver.

5. The system of claim 1, further comprising:
a precision linear stage to accurately position the ultrasonic transducer substantially proximal to the ocular tissue.

6. The system of claim 1, wherein the ocular tissue is corneal tissue.

7. The system of claim 1, wherein the ocular tissue is scleral tissue.

8. The system of claim 1, wherein a wave propagation model is developed and biomechanical properties are determined.

9. The system of claim 1, wherein the measured biomechanical properties are density, stiffness, thickness, and longitudinal modulus.

10. The system of claim 9, wherein corneal speed of sound can be determined by the determined biomechanical properties of stiffness and density.

11. The system of claim 1, wherein the ultrasonic transducer has an acoustic intensity of less than 3 mw/cm².

12. The system of claim 1, further comprising:
an output display to display the ultrasonic reflections and the reflection spectra.

13. A method for non-invasively ultrasonically measuring biomechanical properties of ocular tissue in vivo, the method comprising:
centering a ultrasonic transducer substantially proximate to a center of the apex of the ocular tissue;
obtaining ultrasonic reflections of the ocular tissue from ultrasonic incident waves sent by the ultrasonic transducer;
converting the ultrasonic reflections into reflection spectra by a processor; and
determining biomechanical properties of the ocular tissue based on the characteristics of the reflection spectra.

14. The method of claim 13, further comprising:
grossly positioning the ultrasonic transducer substantially proximal to the ocular tissue before activating the ultrasonic transducer.

15. The method of claim 13, further comprising:
developing a wave propagation model to determine biomechanical properties by simulating ultrasound propagation in ocular tissue in vivo.

16. The method of claim 15, further comprising:
fitting simulated reflection curves from the wave propagation model to the reflection spectra from the processor.

17. The method of claim 16, wherein the fitting is performed using a Levernberg-Marquardt, nonlinear, least square algorithm.

18. The method of claim 13, wherein the ultrasonic reflections are converted to reflection spectra using fast Fourier Transformation.

19. The method of claim 13, further comprising:
calculating speed of sound of the ocular tissue from the determined biomechanical properties.

20. The method of claim 13, further comprising:
applying a saline-filled eye cup to the ocular tissue.

21. A method for non-invasively ultrasonically measuring biomechanical properties of ocular tissue in vivo, the method comprising:
centering a ultrasonic transducer substantially proximate to a center of the apex of the ocular tissue;
obtaining ultrasonic reflection waves of the ocular tissue from ultrasound incident waves sent by the ultrasonic transducer;
converting the ultrasonic reflection waves into reflection spectra by a processor; and
determining biomechanical properties of the ocular tissue based on the characteristics of the reflection spectra based on a wave propagation model for ocular tissue.
